(19) United States
(12) Patent Application Publication
(10) Pub. No.: US 2013/0345554 A1
(43) Pub. Date: Dec. 26, 2013

(54) Methods for recanalization of vessels

(71) Applicant: Baylis Medical Company Inc., Mississauga (CA)

(72) Inventors: Gareth Davies, Toronto (CA); Ramsey Leung, Toronto (CA)

(21) Appl. No.: 13/912,244

(22) Filed: Jun. 7, 2013

Related U.S. Application Data
(60) Provisional application No. 61/657,371, filed on Jun. 8, 2012.

(51) Int. Cl. A61B 6/12; A61B 17/3205; A61B 6/00

(52) U.S. Cl. CPC A61B 6/12; A61B 6/485; A61B 17/32056; A61B 6/4441
USPC 600/424

(57) ABSTRACT

A method for advancing a medical instrument along a desired trajectory or path in one dimension while remaining within a particular area defined in an orthogonal dimension. For example, a method of traversing an occlusion within a body vessel using a medical instrument, the method comprising steps of positioning a target object at a first side of the occlusion, imaging the target object along an imaging plane substantially parallel to a longitudinal axis of the occlusion and advancing the medical instrument within the body vessel through an opposite side of the occlusion, thereby traversing the occlusion.

This application claims the benefit of U.S. Provisional Patent Application Ser. No. 61/657,371, filed on Jun. 8, 2012, incorporated herein by reference in its entirety.

### TECHNICAL FIELD

The disclosure relates to methods for treating occlusions in body vessels. More specifically, the disclosure relates to methods for recanalizing body vessels.

### BACKGROUND OF THE ART

Various forms of wire snares and other such instruments have been used in the past to guide certain medical procedures in the human body. For example, “A New Pathway for Central Venous Occlusion” by McGuckin et al., Endovascular Today, July 2009, “Radiofrequency Perforation in the Treatment of Congenital Heart Disease” by Benson et al., Catheterization and Cardiovascular Interventions, 2002 and “Central Venous Occlusions From Simple Solutions to Advanced Techniques” by Rajan et al., Endovascular Today, June 2012, each describe the use of a snare as a target for a wire or other medical instrument traversing an anatomical structure or vessel occlusion within a human body. Generally, the procedures performed using snares and the like for guidance required some form of medical imaging able to capture images along orthogonal planes, such as biplanar fluoroscopy. Such imaging methodologies may not be practicable in certain situations depending, for example, on equipment availability and the particular anatomy being imaged, amongst other factors.

### SUMMARY OF THE DISCLOSURE

In some patients, for example patients having occlusions in the central veins, such as are often seen in patients undergoing dialysis, it may be difficult to employ biplanar orthogonal fluoroscopy (i.e. imaging along two orthogonal planes) due to the numerous bony structures located in the vicinity of the imaging target site. In such situations, single plane fluoroscopy is often used to better direct the x-ray beam in order to avoid those bony structures and other obstructions to the extent possible.

The present inventors have discovered a method of traversing occlusions or other body structures using single plane imaging techniques, such as fluoroscopy. In some embodiments of the invention, a snare or other target object, having a substantially two-dimensional shape on a particular plane, may be viewed along the longitudinal axis of the occlusion or vessel (otherwise known as a ‘gun barrel’ or ‘down the barrel’ approach) in order to define a two dimensional target area. A medical instrument, such as a mechanical or radiofrequency-based guidewire, is positioned on the side of the occlusion opposite the target object and is advanced through the occlusion. As the medical instrument is advanced, an image of the disease area may be obtained (either concurrent with the advancement or temporally separate from the advancement) along the longitudinal axis of the occlusion/vessel in order to allow a user to visualize a radial position of the medical instrument relative to the target area and to thereby make any necessary adjustments to maintain the position of the medical instrument within the boundary of the target area defined by the target object. This helps to ensure that the medical instrument is not advanced along a trajectory that will take the medical instrument outside of the body vessel.

In some embodiments of the present invention, further enhancements to the safety and efficacy of the procedure may be obtained by rotating the single plane imager to define a volumetric roadmap through which the medical instrument should be advanced to avoid exiting the true vessel lumen.

Thus, in one broad aspect, some embodiments of the present invention allow a medical instrument to be advanced along a path within a patient's body, such as a blood vessel, while helping to ensure that the device remains located within a designated or desirable area as it is advanced. For example, certain embodiments of the present invention help to ensure that a wire, such as a guidewire, that is inserted within a body vessel to, for example, cross an occlusion within the vessel, remains within the true lumen of the vessel as it is advanced. Such embodiments may be particularly useful where the path of least resistance may cause the medical instrument to deviate from the desired trajectory (for example, to leave the true lumen and possibly perforate the vessel wall).

In a specific example of this broad aspect, an embodiment of the present invention includes a method of traversing an occlusion within a body vessel using a medical instrument, the method comprising: positioning a target object at a first side of the occlusion; imaging the target object along an imaging plane substantially parallel to a longitudinal axis of the occlusion; and advancing the medical instrument within the body vessel through an opposite side of the occlusion thereby traversing the occlusion.

In accordance with a second broad aspect, embodiments of the present invention comprise a method for advancing a medical instrument along a desired trajectory. The method comprises visualizing a target object in a manner which allows for a view of the object in two dimensions to be determined and determining/maintaining a position of a medical instrument within a boundary defined by the target object in a first plane while advancing the medical instrument along a different plane (e.g. orthogonal).

In a third broad aspect, embodiments of the present invention comprise a method for advancing a medical instrument through a conduit along a desired trajectory. The method comprises visualizing a target object in a manner which allows for a view of the object in two dimensions to be determined and determining/maintaining a position of a medical instrument within a boundary defined by the target object while advancing the medical instrument.

In a fourth broad aspect, embodiments of the present invention comprise a method for advancing a medical instrument towards a desired target site. The method comprises visualizing a target object in a manner which allows for a view of the object in two dimensions to be determined and determining/maintaining a position of a medical instrument within a boundary defined by the target object while advancing the medical instrument.

In a fifth broad aspect, embodiments of the present invention comprise a method for advancing a medical instrument along a desired trajectory/path in one dimension while remaining within a particular area defined in the orthogonal dimensions. The method comprises visualizing a target object in a manner which allows for a view of the object in two dimensions to be determined and determining/maintaining a position of a medical instrument within a boundary defined by the target object while advancing the medical instrument.

In a sixth broad aspect, embodiments of the present invention comprise a method for advancing a medical instrument along a desired trajectory/path in one dimension while remaining within a particular area defined in the orthogonal dimensions. The method comprises defining a two dimensional boundary/area/region based on a target object positioned along a desired trajectory/path of a medical instrument and determining/maintaining a position of a medical instrument within the boundary defined by the target object while advancing the medical instrument.

As a feature of these aspects, the target object may be visualized in a “gun barrel” approach by imaging along a plane substantially parallel to a longitudinal axis of the occlusion, whereby a two dimensional view of the target object may be obtained using a single exposure/image capture.

As an additional feature, the target object may be a snare or any other object capable of presenting/providing a two dimensional image/projection using a medical imaging modality, such as x-ray fluoroscopy, ultrasound (including echocardiography) or magnetic resonance imaging. Alternatively, the target object may otherwise visually define an area within which the medical instrument may be guided.

In a seventh broad aspect, a system for traversing an occlusion within a body vessel is described, the system comprising: a medical imager operable to obtain an image along a plane substantially parallel to an occlusion within a body vessel; and a target object configured to present a substantially closed loop when positioned at the occlusion and when visualized using the medical imager along the plane.

As a feature of this seventh broad aspect, the target object is a snare.

### BRIEF DESCRIPTION OF THE DRAWINGS

In order that the invention may be readily understood, embodiments of the invention are illustrated by way of examples in the accompanying drawings, in which:

FIG. 1 is a flow chart showing steps that may be performed under embodiments of the present invention;

FIG. 2 illustrates an example of a treatment site for employing an embodiment of the present invention;

FIGS. 3A-3C are ‘gun barrel’ views illustrating various embodiments of target objects;

FIGS. 4A and 4B are illustrations of boundaries used to determine three-dimensional roadmaps under embodiments of the present invention;

FIG. 5 shows a target site at a Left Brachiocephalic Vein;

FIG. 6 shows entry points for inserting devices for accessing the Left Brachiocephalic Vein;

FIGS. 7A-7C illustrate an anterior-posterior view of a medical instrument and target object;

FIGS. 8A-8C illustrate a Right Anterior Oblique view of a medical instrument and target object;

FIGS. 9A-9C illustrate a Left Anterior Oblique view of a medical instrument and target object;

FIG. 10A show images of upper extremity (brachial approach) and central (femoral approach) venograms;

FIGS. 10B-10C show images of right anterior-oblique (RAO) and left anterior-oblique (LAO) views; and

FIGS. 10D-10E show images of a snare capture of a medical instrument.

### DETAILED DESCRIPTION

With specific reference now to the drawings in detail, it is stressed that the particulars shown are by way of example and for purposes of illustrative discussion of certain embodiments of the present invention only. Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Various medical instruments may be utilized within embodiments of a method of the present invention. For example, if an embodiment of the present invention is used in the context of crossing an occlusion within a body vessel, the medical instrument may comprise a guidewire. In some embodiments, the guidewire may be a radiofrequency-based guidewire such as is described in one or more of: U.S. patent application Ser. No. 11/627,406, U.S. patent application Ser. No. 11/520,754, U.S. patent application Ser. No. 11/265,304, U.S. Pat. No. 7,048,733, U.S. application Ser. No. 10/760,479, U.S. application Ser. No. 10/666,288 and/or U.S. Pat. No. 7,112,197. All of these patents and patent applications are hereby incorporated by reference in their entirety.

It should be noted that the term ‘occlusion’, as used herein, may refer, for example, to any one of a total occlusion, a partial occlusion or a stenosis.

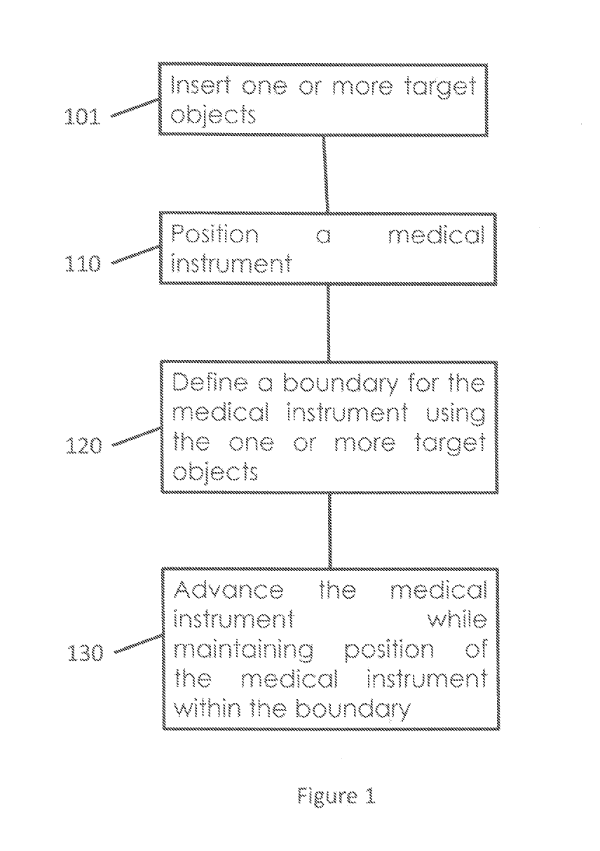

As shown in the flow chart of FIG. 1, some embodiments of a method the present invention generally include the following:

Deriving/defining (120) a (target) boundary for a medical instrument using one or more target objects.

Advancing (130) the medical instrument (towards one of the target objects) while maintaining position of the medical instrument within the boundary.

Prior to performing the aforementioned steps of an embodiment of the present invention, a user would typically also do the following:

Inserting (101) one or more target objects into a treatment site within a patient's body.

Positioning (110) a medical instrument at the treatment site.

Some embodiments include a step of characterizing/defining a path along which the medical instrument should be advanced.

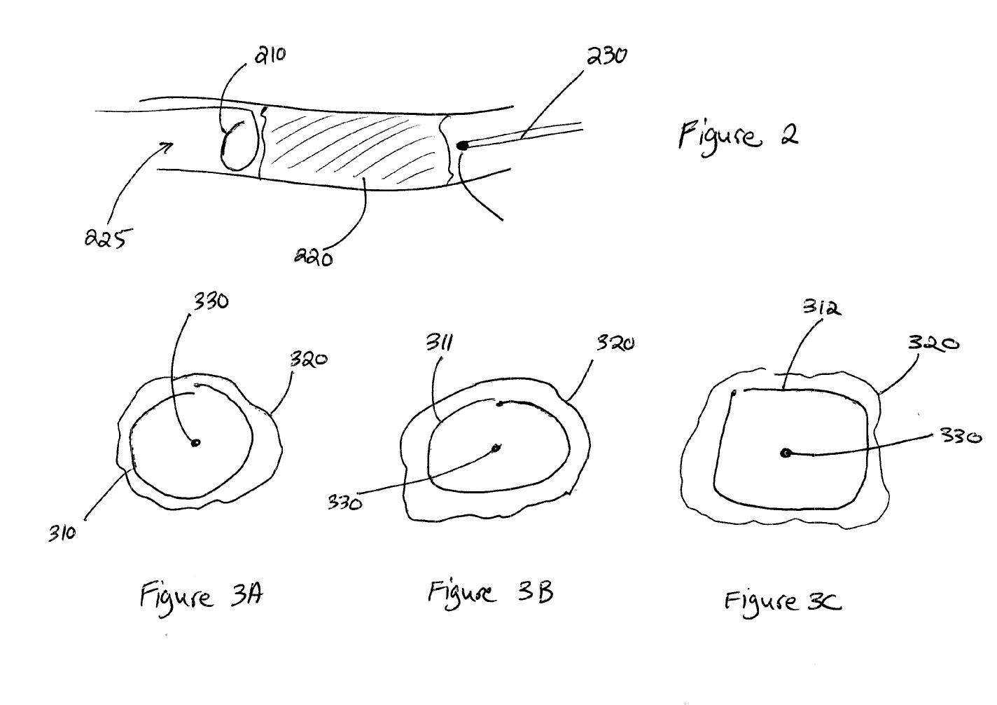

The target object(s) may be, for example, a snare or other object able to provide a two-dimensional boundary using a medical imaging modality. In the case of the snare, using x-ray fluoroscopy in either a “gun barrel” approach, whereby an image is captured while looking ‘down the barrel’ through the snare along a plane substantially parallel to a longitudinal axis of the occlusion, or using two orthogonal views, will enable determination of the two-dimensional boundary. FIG. 2 illustrates a snare 210 positioned at one end (for example a distal end, where distal is defined relative to a user) of an occlusion 220. In alternative embodiments, a snare or other target object may be placed on the opposite (for example, proximal) side of the occlusion for improving alignment and determining an optimal path for the medical instrument 230, as described further hereinbelow.

In some embodiments, the treatment site comprises an occluded portion of a blood vessel 225 or other body conduit. FIG. 2 illustrates a typical chronic total occlusion of a blood vessel. In alternative embodiments, the treatment site comprises another location within a patient's body where it is desirable to advance a medical instrument from a first location to a second location where direct visualization is difficult, impossible or impractical.

The medical instrument 230 may be, for example, a guidewire used to traverse occlusion 220. The guidewire may be a mechanical guidewire or may incorporate a form of energy delivery such as radiofrequency or microwave electrical energy, laser or other optical energy and/or ultrasound or other vibrational energy. In alternative embodiments, the medical instrument may include another tool or implement which may be advanced through the patient's body at the treatment site.

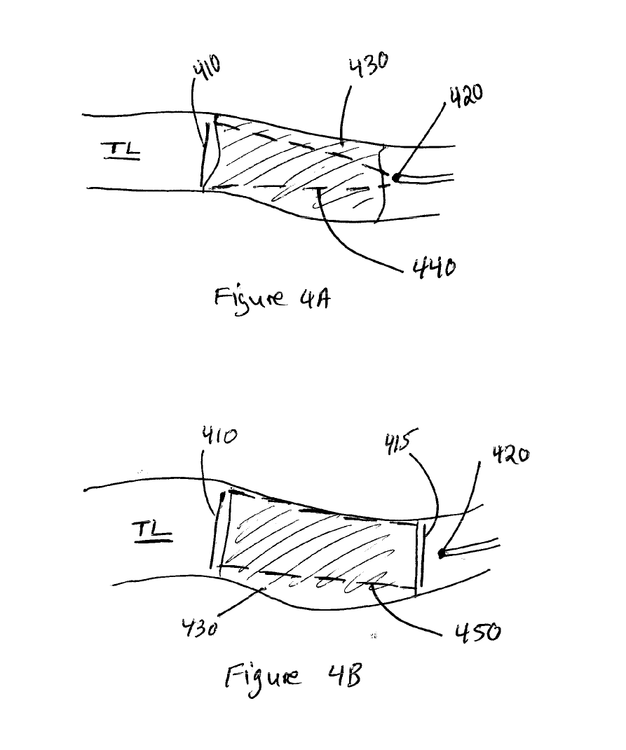

FIGS. 3A-3C illustrate examples of boundaries that may be visible using, for example, a “gun barrel view”. FIG. 3A shows a substantially circular target object 310 (i.e. shaped like a circle) positioned within a body conduit 320. Medical instrument 330 is shown as a dot, indicating that is substantially aligned with the target object 310. FIGS. 3B and 3C show substantially oval 311 and square-shaped 312 target objects, respectively.

As noted hereinabove, some embodiments of the present invention include a step of characterizing/defining a path along which the medical instrument should be advanced. This may be desirable, for example, to ensure that the medical instrument remain within a true lumen of a body conduit such as a blood vessel.

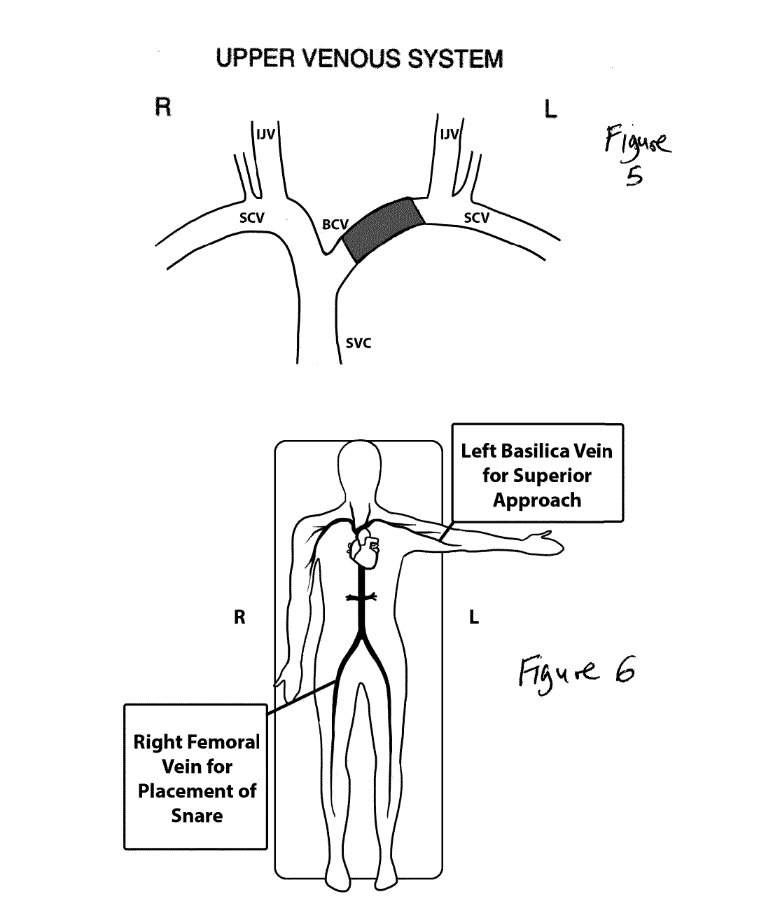

FIG. 4A shows an exemplary illustration of a view along a single plane (for example, the anterior-posterior [AP] plane) indicating a projection along that plane of a target object 410 and a medical instrument 420. The target object 410 and medical instrument 420 are positioned at substantially opposite sides of occlusion 430. Dashed lines 440 define a triangle in this single plane showing the boundaries/edges, in this view, within which the medical instrument should be constrained in order to ensure that it remain within the true lumen (TL) of the vessel. Obtaining a similar image in an orthogonal plane allows for a determination/construction of a 3-dimensional volume or map (effectively by rotating the single plane boundary along its longitudinal axis producing, in this case, a cone-shaped volume) within which the medical instrument should be advanced. In some embodiments of the invention, views such as those described may be obtained prior to advancing medical instrument 420 into the occlusion so as to define a desired path/trajectory for medical instrument 420.

FIG. 4B illustrates two target objects, 410 and 415, positioned at substantially opposing sides of occlusion 430. In this scenario, the single-plane boundary 450 defined by the two target objects appears as a rectangle and the 3-dimensional volume determining by obtaining an orthogonal view will comprise a cylinder.

In some embodiments, the orthogonal views are obtained along the Left Anterior Oblique and Right Anterior Oblique planes.

In some embodiments, the volume maps described above are reviewed or consulted during the procedure to ensure that the medical instrument remains within the true lumen. This may involve regenerating or obtaining images substantially identical to those obtained prior to advancing the medical instrument and registering/comparing the current position of the medical instrument relative to the original boundaries obtained initially. This step may be performed one or more times during the course of the treatment procedure. In some embodiments, a user may advance the medical instrument in small increments (for example, about 1 mm to about 5 mm) while checking the position of the device prior to each subsequent advancement. Alternatively, a user may document the original boundaries on a display (for example, by using markers or other writing implements to draw an overlay of the boundary on a screen) whereby the position of the medical instrument may be compared to the documented boundaries to ensure that the medical instrument remains within the desired volume/area.

Steps for practicing one particular embodiment of a method of the present invention are described herein below.

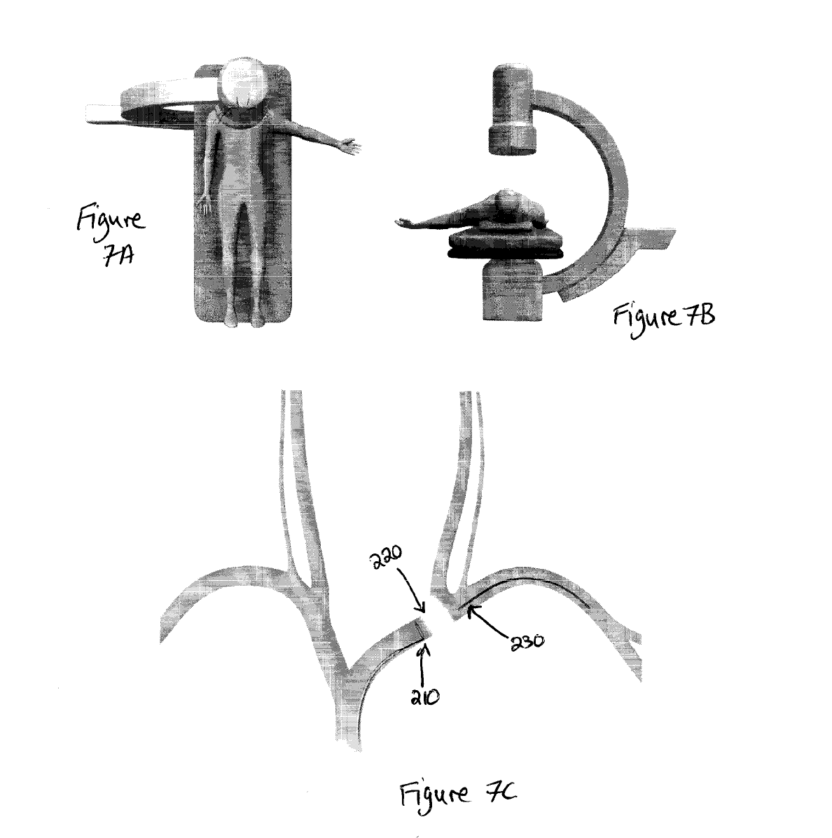

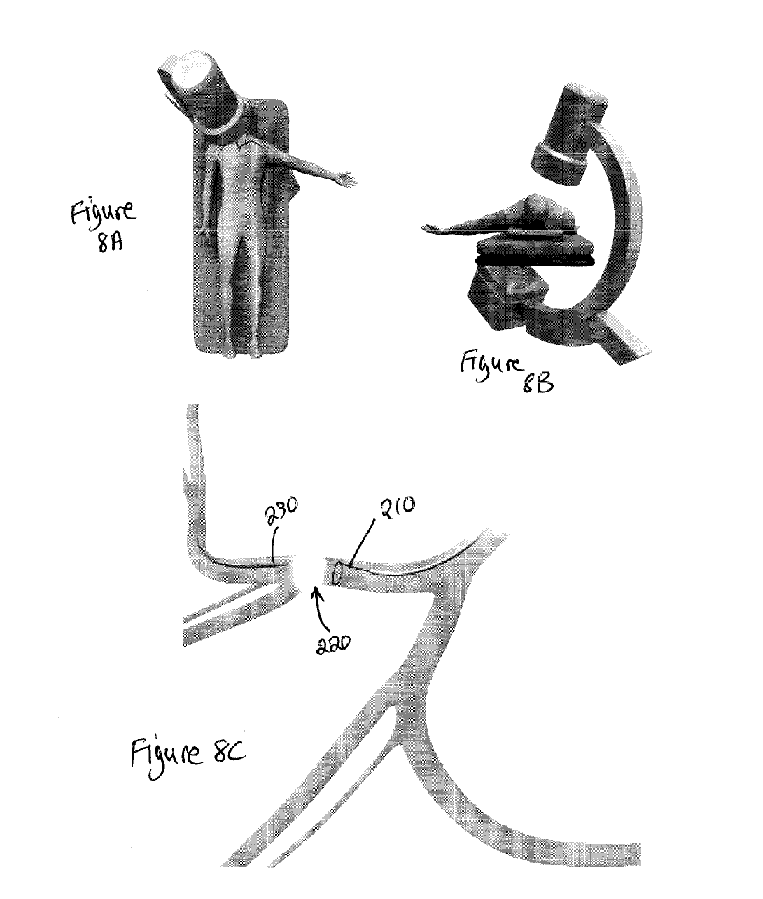

A radiopaque device is placed at the target, for example the Left Brachiocephalic Vein, as illustrated in FIG. 5. Typically, where the target is within a vessel, the target will define or outline the acceptable landing zone or destination for the medical instrument. An example of a suitable radiopaque device would be a radiopaque wire that substantially loops around the circumference of a vessel at the end of a CTO, such as a snare. Alternatively, a stored angiogram may be used to define the acceptable landing zone. The snare may be inserted via the Right Femoral Vein, as illustrated in FIG. 6.

Advance the medical instrument, such as a radiopaque tipped guidewire, to the start of the lesion (i.e. where mechanical resistance is felt, impeding further advancement of the guidewire). As illustrated in FIG. 6, the medical instrument may be inserted via the Left Basilica Vein to reach the target treatment site.

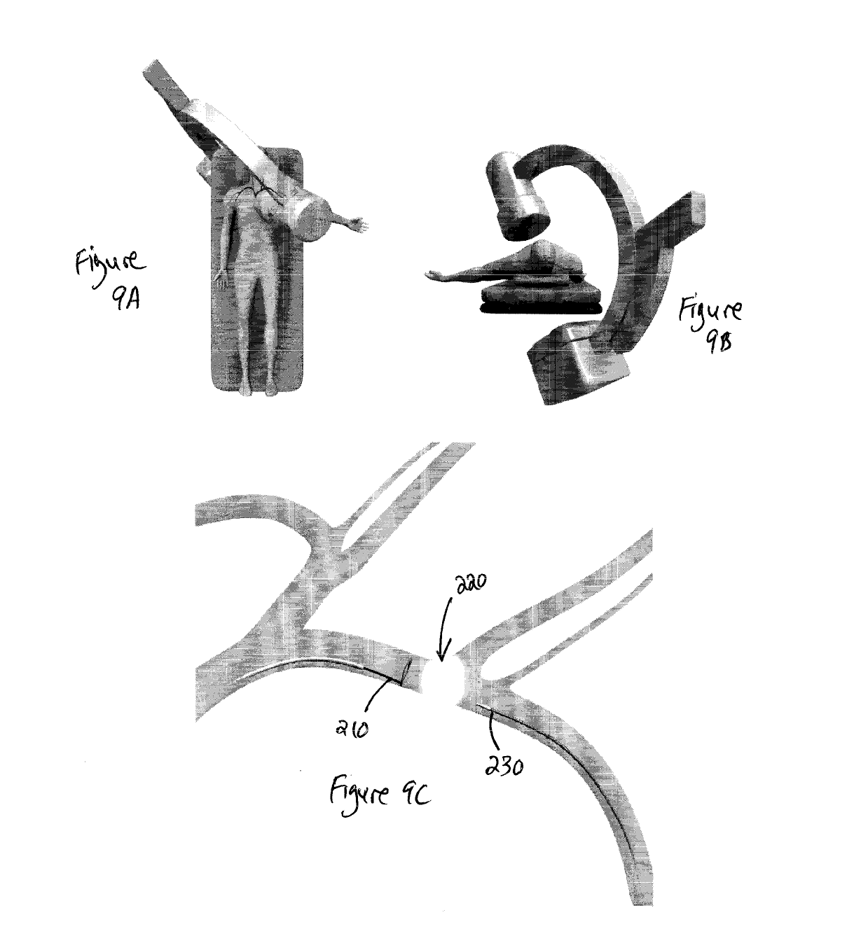

As described hereinabove, an initial ‘roadmap’ may be obtained under an AP view, an example of which is shown in FIGS. 7A-7C. FIGS. 7A and 7B illustrate the position of the C-arm fluoroscopy instrument while FIG. 7C shows an exemplary image as may be obtained using this C-arm orientation.

Through consulting 3 imaging planes, for example, determine the next small incremental target (for advancement) and ensure that the wire is oriented in that direction. It may be desirable to ensure that the snare is positioned substantially perpendicular to the vessel wall and substantially flush with the end-cap of the occlusion. The 3 planes consist of:

Anterior-Posterior, as shown in FIGS. 7A-7C.

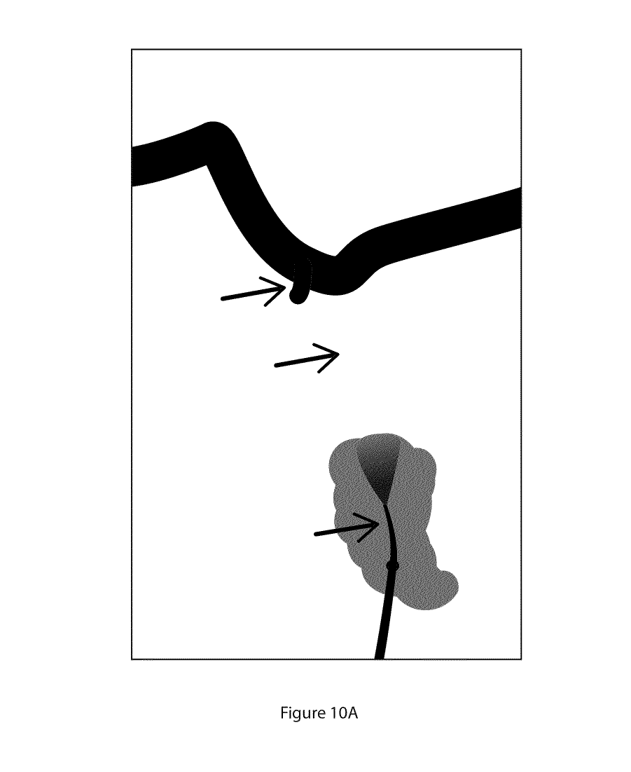

Right Anterior Oblique (ideally the RAO and LAO will be offset by 90 degrees, however this is not always possible depending on anatomy and the mobility of the C-arm or other imaging device), as shown in FIGS. 8A-8C.

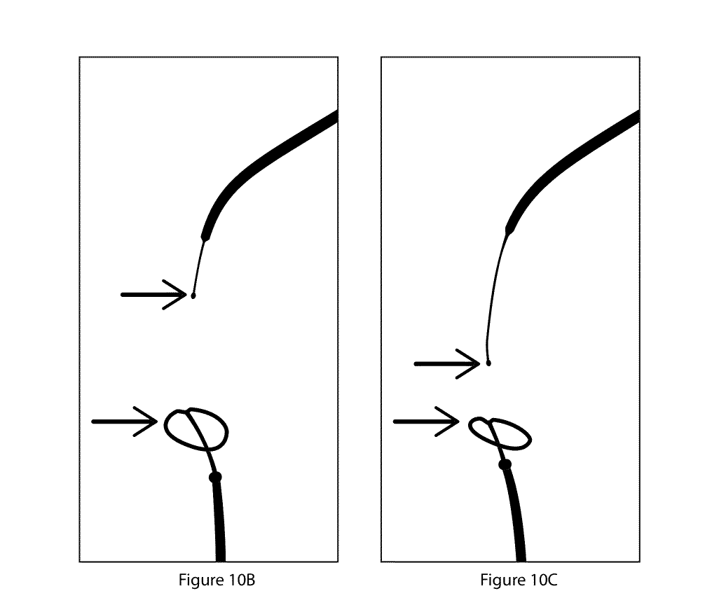

Left Anterior Oblique, shown in FIGS. 9A-9C.

In alternative embodiments, as described hereinabove, another view referred to as “Down the barrel” or a “gun barrel” view (a view where we are looking through the snare or loop and at the wire tip, for example in a plane substantially parallel to a longitudinal axis of the occlusion. As evident by the fact that the tip will fall within the loop.) may be used. In such an embodiment, it may suffice to consult the gun barrel view, possibly in conjunction with the Anterior-Posterior view, without requiring the RAO and LAO views.

Advance, possibly via delivering radiofrequency (RF), to the incremental/intermediate target site (i.e. point of advancement) while continuing to monitor the fluoroscopy or other imaging modality, for example under the “gun barrel” or AP views. Advancement as described herein may be implements using a number of discrete steps, whereby the medical instrument/wire is advanced by a relatively small amount at a time.

Repeat the imaging consultation step (i.e. imaging the target object and/or treatment or disease site), identification of next incremental target (i.e. next point of advancement), wire orientation and advancement, etc.

Continue until the wire is seen to pass through the snare/loop.

In some embodiments, 2 snares may be positioned at the target site, one slightly offset form the other, for the purpose of aiding alignment of the “gun barrel” view. Alternatively, 1 snare may be positioned distal to the occlusion while another snare is positioned proximal to the occlusion. Alignment of the snares helps define the path along which the guidewire should be advanced. In either of these embodiments, the step of visualizing or imaging the target object comprises imaging both the target object (i.e. the first snare positioned at the target site or first side of the occlusion) as well as the second target object (either the second snare positioned at the target site/first side or, alternatively, the second snare positioned at the opposite side).

In some embodiments, a user may measure the longitudinal distance from the wire tip to the next incremental target, i.e. a desired point of advancement, prior to advancement and may place a depth or physical marker (such as a torque device) at the wire such that advanced of the wire beyond this point is impeded. This may facilitate use of the gun barrel view and may assist in overcoming the lack of feedback on how far he has advanced the wire.

### Case Study 1

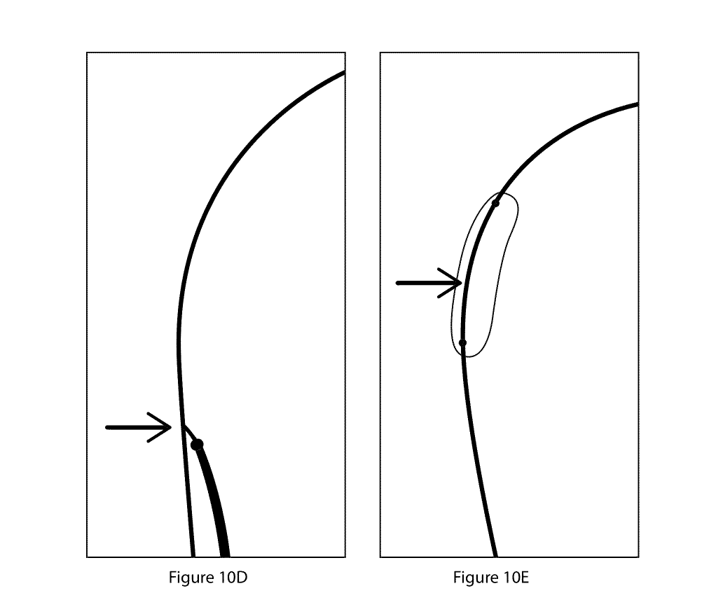

Under fluoroscopic guidance, simultaneous upper extremity (brachial approach) and central (femoral approach) venograms were used to define the central venous occlusion segment (length, diameter of the venous stumps) and to determine the approach (right versus left arm) and direction of wire advancement (cranio-caudal versus caudo-cranial) (FIG. 10A). The RF wire was advanced within a 5-Fr catheter until the occlusion was reached. A 10 mm snare was placed adjacent to the lesion in the opposite venous side through a 5-Fr diagnostic catheter for use as a target.

Alignment between the RF wire tip and the snare was checked in antero-posterior (AP), right anterior-oblique (RAO) and left anterior-oblique (LAO) views before delivery of RF energy (FIGS. 10B-C). RF energy was delivered while the wire was gently advanced a few millimeters at a time. Whenever the RF wire puncture was inadequate, a curved RF wire or diagnostic catheter was used to approach the occlusion on a different angle and RF was again delivered.

Once the lesion was crossed, the snare catheter was used to assist a 125 cm 5-Fr catheter that was advanced “through-and-through” and the RF wire was exchanged for a 260 cm stiff 0.035″ wire (FIGS. 10D-E). Under the “body floss” technique, 4 mm balloon angioplasty pre-dilation was followed by primary stent placement in all patients. In two of the initial cases, self-expandable covered stents were placed in the subclavian vein. For the remainder of the subjects, self-expandable bare stents were placed in the subclavian vein and post-dilated, and balloon-expandable bare stents were placed in the superior vena cava (SVC) and brachiocephalic vein. Dilation diameters ranged from 9-12 mm, depending on the anatomy. Heparin was not administered for acute thrombosis prevention. Antiplatelet therapy (81 mg of acetylsalicylic acid) was prescribed to all patients, unless the patient had a contra-indication or was under anticoagulant therapy.

The embodiments of the invention described above are intended to be exemplary only. The scope of the invention is therefore intended to be limited solely by the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

We claim:

1. A method of traversing an occlusion within a body vessel using a medical instrument, the method comprising:
positioning a target object at a first side of the occlusion;
imaging the target object along an imaging plane substantially parallel to a longitudinal axis of the occlusion; and
advancing the medical instrument within the body vessel through an opposite side of the occlusion, thereby traversing the occlusion.

2. The method of claim 1, wherein the steps of imaging and advancing are performed substantially concurrently.

3. The method of claim 1, wherein the steps of imaging and advancing are substantially separated in time.

4. The method of claim 1, wherein the step of imaging comprises imaging both the target object as well as the medical instrument along the imaging plane substantially parallel to the longitudinal axis of the occlusion.

5. The method of claim 4, further comprising adjusting an orientation of the medical instrument in response to the step of imaging, for ensuring that the medical instrument remains within a lumen of the body vessel during the step of advancing.

6. The method of claim 1, wherein the step of advancing comprises advancing the medical instrument in a number of discrete steps to traverse the occlusion.

7. The method of claim 6, wherein the step of imaging is repeated between each of the discrete steps of advancing the medical instrument.

8. The method of claim 7, further comprising adjusting an orientation of the medical instrument in response to the step of imaging, for ensuring that the medical instrument remains within a lumen of the body vessel during each of the discrete steps of advancing.

9. The method of claim 1, wherein the target object is configured to present a two-dimensional shape when visualized during the step of imaging.

10. The method of claim 9, wherein the two-dimensional shape comprises a substantially closed loop.

11. The method of claim 10, wherein the two-dimensional shape comprises a circle.

12. The method of claim 10, wherein the two-dimensional shape comprises an oval.

13. The method of claim 10, wherein the two-dimensional shape comprises a square.

14. The method of claim 11, wherein the target object comprises a snare.

15. The method of claim 1, further comprising a step of positioning a second target object at the first side of the occlusion, the second target object being offset from the target object, and wherein the step of imaging comprises imaging both the target object as well as the second target object.

16. The method of claim 1, further comprising a step of positioning a second target object on the opposite side of the occlusion and wherein the step of imaging comprises imaging both the target object as well as the second target object.

17. The method of claim 1, further comprising measuring a longitudinal distance from a tip of the medical instrument to a desired point of advancement, and placing a marker along the medical instrument for impeding advancement of the medical instrument beyond the desired point of advancement.

18. A system for traversing an occlusion within a body vessel, the system comprising:
a medical imager operable to obtain an image along a plane substantially parallel to an occlusion within a body vessel; and
a target object configured to present a substantially closed loop when positioned at the occlusion and when visualized using the medical imager along the plane.

19. The system of claim 18, wherein the target object is a snare.
